(19) United States
(12) Patent Application Publication
(10) Pub. No.: US 2010/0056977 A1
(43) Pub. Date: Mar. 4, 2010

(54) Trans-corneal shunt and method

(76) Inventor: Thaddeus Wandel, Croton, NY (US)

Correspondence Address: ROBERT NATHANS, 36 STAG DRIVE, BILLERICA, MA, 01821, US

(21) Appl. No.: 12/229,960

(22) Filed: Aug. 26, 2008

(51) Int. Cl. A61M 1/00

(52) U.S. Cl. 604/8

(57) ABSTRACT

A method for relieving pressure within the eye in the treatment of glaucoma comprising inserting an extremely thin flat trans-corneal drainage shunt, having no detrimental cap or footplate, within a self-sealing incision made by a keratome knife within the cornea at the limbus, the drainage shunt having extremely small pores formed therein for the controlled transport of aqueous fluid from an anterior portion of the eye to the tear film on the surface of the eye while deterring bacteria from entering the eye, and fixing the position of the trans-corneal shunt in position within the cornea by means of a suture. The shunt can be tapered for preventing an unacceptable degree of intrusion of the shunt into the anterior portion of the eye. The trailing edge portion of the shunt is wide relative to other portions of the shunt, thereby facilitating grasping of the shunt when desired.

### BACKGROUND OF THE INVENTION

Lippman, U.S. Pat. No. 5,073,163 issued Dec. 17, 1991 drains the aqueous humor under the conjunctiva, not into the tear film on the surface of the cornea as in the present invention. No advantage is seen in Lippman's shunt over traditional shunts which do the same.

Regarding Brown U.S. Pat. No. 5,743,868 issued Apr. 28, 1998; his shunt, since it has no cap AND cannot be guaranteed over time to be held in the cornea, and thus has an unacceptable risk of intrusion into the anterior chamber through it's round hole in the cornea.

Regarding Ungerleider, U.S. Pat. No. 4,936,825 issued Jun. 26, 1990: his device penetrates the cornea in a loop. The device then has the potential to touch the iris with the loop portion that is inside the eye. This will create a chronic inflammatory response. This chronic inflammation can destroy the eye. The loop which penetrates the cornea on the surface can move with eye blink and weaken the area around the cornea-device junction. This will allow bacteria to enter and potentially destroy the eye.

Regarding Simon U.S. Pat. No. 7,207,965 issued Apr. 24, 2007, his non-flat shunt was created to shunt aqueous humor from the anterior chamber to a space inside the eye between the sclera and the choroid. Such insertion can induce bleeding. Also, movement of the device with eye blink can create chronic inflammation.

Ahmed shunts are also known in the art having large flat plates placed over the sclera with a tube going into the anterior chamber of the eye. Such Ahmed shunts are mentioned in col. 2 of U.S. Pat. No. 6,544,208 to Ethier et al., issued Apr. 8, 2003.

Wandel (the present inventor) U.S. Pat. No. 5,807,302 issued Sep. 15, 1998 discloses a trans-corneal shunt to be discussed in detail below.

### BRIEF SUMMARY OF PREFERRED EMBODIMENTS OF THE INVENTION

A thin flat trans-corneal shunt, having no cap or footplate, is inserted into the cornea through a self-sealing incision made by a keratome surgical knife. Extremely small tunnels or pores are formed within the thin flat trans-corneal shunt for transporting aqueous fluid from the anterior of the eye to the tear film on the surface of the eye while deterring bacteria from entering the eye. Fixation means are provided for maintaining the shunt in position upon the cornea by enabling a suture to co-act with a hole(S) or notch formed within the shunt. Additionally the shunt can be tapered for preventing an unacceptable degree of intrusion of the shunt into the anterior portion of the eye. Also, the trailing edge portion of the shunt is wide relative to other portions of the shunt, thereby facilitating grasping of the shunt when desired for removal from the cornea.

### BRIEF DESCRIPTION OF THE DRAWINGS

Other features of the invention may become more apparent upon study of the following detailed description taken in conjunction with the drawings in which:

FIG. 1 discloses the shunt positioned within the cornea near the limbus;

FIG. 2 shows a side view of the shunt;

FIG. 2a shows a top view of the shunt showing the fluid transporting tunnels;

FIG. 3 shows a notch and hole either of which can receive a suture for maintaining the position of the shunt within the cornea.

### DETAILED DESCRIPTION OF PREFERRED EMBODIMENTS OF THE INVENTION

In the aforesaid patent issued to the present inventor Tad Wandel, U.S. Pat. No. 5,807,302, and incorporated by reference herein, the shape of the '302 shunt is cylindrical and has a flange or collar 30a flush with the outer corneal surface 1a and a footplate or collar 30b engaging the inner surface of the cornea. The shunt was round and was easily inserted into the thin and elastic corneas of pigs and rabbits through a linear incision of about 2 mm into the cornea from the limbus (the junction of the clear cornea with the white shell of the eye, the sclera). The animal corneas were found to be very uniform in thickness so that two different lengths of the shunt fit all animals. The seal around the shunt was leak poof without sutures. On removal, the round hole closed spontaneously or needed a single suture to close it.

In contrast, in humans, the corneas were found to be not very elastic, and their thicknesses varied much more than animals. Placement of the '302 shunt resulted in leaks around the shunt which were hard to close even with sutures. When the shunts were removed, the round hole was difficult to close with multiple sutures and resulted in a lot of cornea distortion. Since the thicknesses of the human cornea were varied, it was hard to match the shunt to a person. As a result, some shunts had “play” that resulted in a vertical movement of the shunt in the cornea like a plunger. Because the collar or footplate 30b would hit very sensitive cells on the cornea bottom surface, these cells were injured and produced cornea edema.

Also, the iris was also able to get caught in the bottom foot plate or collar 30b due to the poor fit of the shunt. This can lead to chronic inflammation. Bacteria also had an available ingress into the eye with constant movement of the shunt. It would be hard to make enough different lengths of the shunt to provide perfect fits of shunt to cornea. Even with a round hole made into the cornea, the above problems existed. Because more force was needed to insert the shunt into a human cornea through a snug incision, it was possible to intrude the device into the eye completely through the cornea.

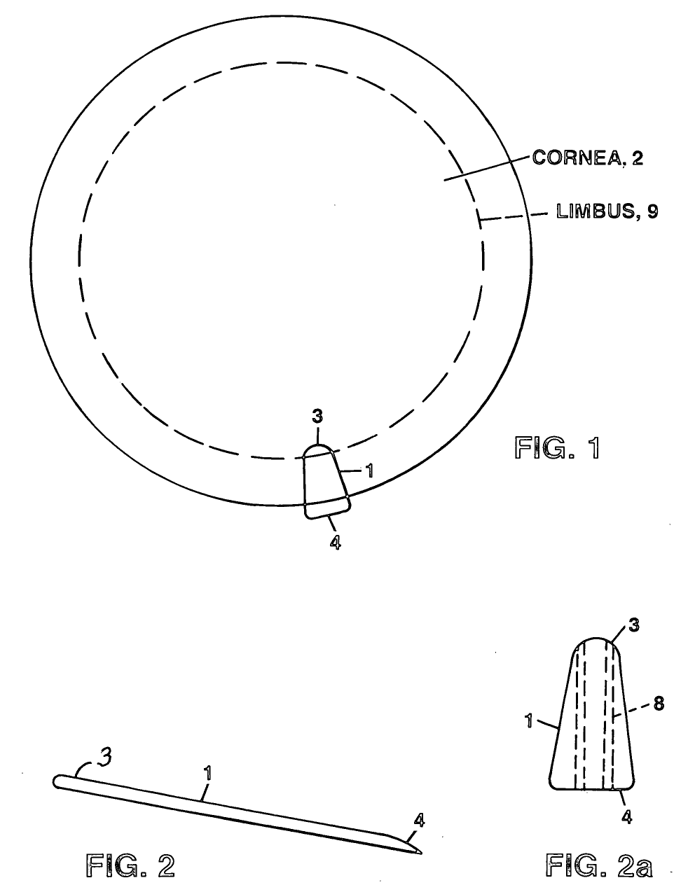

In accordance with the present invention, a thin flat shunt 1 is inserted at the limbus 9 into the cornea 2 as shown in FIG. 1, in a shelved self-sealing incision made by a keratome knife used in cataract surgery, to thus avoid the above stated problems related to the shunt of my '302 patent. The nature of keratome surgical knives and self-sealing incisions made by such knives is discussed in detail in columns 1-4 of U.S. Pat. No. 6,139,559 issued to Nordan et al., issued on Oct. 31, 2000 and incorporated herein by reference.

As shown in FIG. 2, the shunt in one embodiment has a smooth and rounded leading edge 3 to be inserted into the cornea and a smooth sloped trailing edge 4 that rests upon the eye surface. The preferred length between the leading and trailing edges is about 2.5 millimeters (mm). The thickness is about 0.2 mm or 200 microns. The porous extremely small pores or tunnels are formed within the thin flat trans-corneal shunt for transporting aqueous fluid from the anterior of the eye to the tear film on the surface of the eye while deterring bacteria from entering the eye through filtering. Tunnels 8 are shown in FIG. 2a within the shunt 1 of FIG. 1. These pores or tunnels acting as filters are known to workers in the art; see for example column 7 of U.S. Pat. No. 7,207,965 issued to Simon on Apr. 24, 2007. The filter for the shunt preferably will have specifications that exclude particles greater than twenty microns, and provide a controlled flow rate of about two micro-liters per minute. However, it may be that sharp corners in practice may be safer than rounded contours.

The flat shunt 1, because it has one length only, and since it has no previously described cap or footplate, can advantageously be placed into a large variety of cornea thicknesses. It fits a linear incision preferably made by a keratome in an inelastic cornea without a gap, and when removed, will self seal. The iris will not get caught on any edge and the endothelial cells on the back surface of the cornea and will not be in constant irritating contact with the moving foot plate. Without shunt movement, bacteria will be deterred from going around the shunt into the eyeball.

Also, unacceptable degrees of intrusion of the flat shunt into the eyeball can be prevented by making the shunt narrower at the tip 2 going into the eye than the wider base 3, to create a shunt taper, which tapered shunt will be fixed on the surface of the eye. For example the tip 3 can have a width of 1.5 mm and base portion 4 could have a width of 2.0 mm.

The insertion would involve a linear incision with a standard keratome blade (a blade that has a triangle shape) at the limbus, making a 2 mm shelved incision with the tip of the keratome entering the eye but not going all the way through so that a trapezoid shaped path is created. The technique is known to ophthalmologists who do cataract surgery. The device would then slide into the incision controlled by an insertion tool. The tool could be separate from the device or integral to the shunt with a release controlled in both cases by the health care provider. The insertion tool would hold the device at its base and then release it by button for instance.

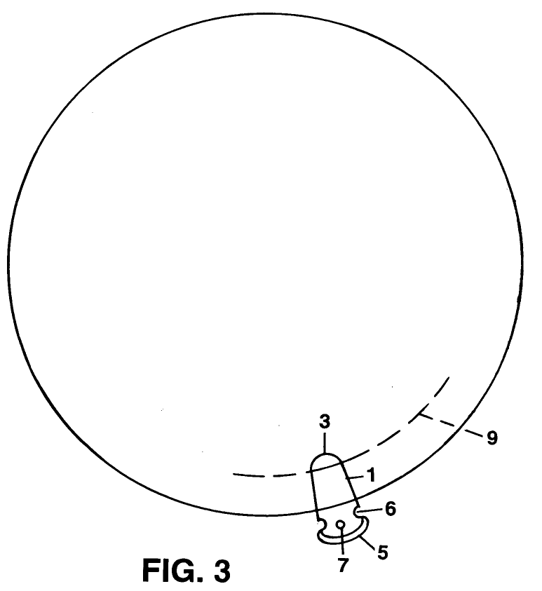

As shown in FIG. 3, provision could be made to fix the shunt to the sclera surface with a 10-0 suture 5 with a groove 6 on the shunt shown in FIG. 3 to hold the suture, or a hole(s) 7 may be provided to pass the suture through. The device would be removed by cutting the suture and gently pulling the shunt out with a forceps.

Alternatively, spur(s) on the shunt close to the base could fix the flat shunt to the sclera. A hook on the edge of the device inside the eye could fix it to the inside cornea edge of the incision facing the iris.

The straight long edges of the device in the cornea stroma could also be shaped to grip the cornea in a passive manner. A fluted shape for instance could be one way for cornea edema to hold the device in position.

Chemicals or materials used to kill bacteria could be imbedded on the surface of the shunt or within the shunt.

While the invention has been described in connection with preferred embodiments, the description is not intended to limit the scope of the invention to the particular forms set forth, but on the contrary, it is intended to cover such alternatives, modifications, and equivalents as may be included within the spirit and scope of the invention as indicated by the language of the appended claims.

I claim:

1. A trans-corneal drainage shunt for relieving pressure within the eye in the treatment of glaucoma comprising:
(a) a thin flat trans-corneal drainage shunt, having no cap or footplate, and having extremely small pores or tunnels formed therein for transporting aqueous fluid from an anterior portion of the eye to tear film on the surface of the eye dimensioned to control the aqueous fluid flow rate while deterring bacteria from entering the eye; and
(b) wherein said thin flat trans-corneal drainage shunt has fixation means for maintaining said shunt in position between said anterior portion of the eye and a surface portion of the eye.

2. The shunt of claim 1 wherein said shunt has a smooth and rounded leading edge to be positioned the eye and a smooth sloped trailing edge to be positioned upon the surface portion of the eye.

3. The shunt of claim 1 wherein said shunt has a thickness of about 200 microns.

4. The shunt of claim 2 wherein said shunt has a thickness of about 200 microns.

5. The shunt of claim 1 wherein said shunt has a length of about 2.5 millimeters.

6. The shunt of claim 2 wherein said shunt has a length of about 2.5 millimeters.

7. The shunt of claim 3 wherein said shunt has a length of about 2.5 millimeters.

8. The shunt of claim 2 wherein said trailing edge portion is relatively wide relative to other portions of said shunt, thereby facilitating grasping of said shunt when desired.

9. The shunt of claim 1 wherein said fixation means includes a hole or notch formed in a trailing edge portion of said shunt for receiving a suture therein.

10. The shunt of claim 2 wherein said fixation means includes a hole or notch formed in the trailing edge of said shunt for receiving a suture therein.

11. The shunt of claim 3 wherein said fixation means consists of a hole or notch formed in the trailing edge of said shunt for receiving a suture therein.

12. A trans-corneal drainage shunt for relieving pressure within the eye in the treatment of glaucoma comprising:
(a) a thin flat trans-corneal shunt having no cap or footplate;
(b) extremely small pores formed within said flat trans-corneal shunt for transporting aqueous fluid from an anterior portion of the eye to tear film on a surface portion of the eye while deterring bacteria from entering the eye; and wherein
(c) said shunt is tapered for preventing an unacceptable degree of intrusion of said shunt into said anterior portion of the eye; and
(d) wherein said shunt has fixation means for maintaining said shunt in position between said anterior portion of the eye and the surface of the eye.

13. The shunt of claim 12 wherein said shunt has a smooth and rounded leading edge portion to be positioned within said anterior portion of the eye and a smooth sloped trailing edge portion to be positioned upon a surface portion of said eye.

14. The shunt of claim 12 wherein said thin flat trans-corneal shunt has a thickness of about 200 microns.

15. The shunt of claim 13 wherein said trailing edge portion is relatively wide relative to other portions of said shunt, thereby facilitating grasping of said shunt when desired.

16. The shunt of claim 12 wherein said fixation means includes a hole or notch formed in the trailing edge of said shunt for receiving a suture therein.

17. The shunt of claim 13 wherein said fixation means includes a hole or notch formed in the trailing edge of said shunt for receiving a suture therein.

18. A method for relieving pressure within the eye in the treatment of glaucoma comprising the steps of:
(a) forming an incision within a cornea portion of the eye;
(b) inserting a thin flat trans-corneal drainage shunt, having no cap or footplate, within said incision, said drainage shunt having extremely small pores formed therein for transporting aqueous fluid from an anterior portion of the eye to the tear film on a surface portion of the eye while deterring bacteria from entering the eye; and
(c) fixing the position of said thin flat trans-corneal shunt in position within said cornea portion between said anterior portion of the eye and a surface portion of the eye.

19. The method of claim 18 wherein said incision is made by a keratome knife, used in cataract surgery, for producing a self-sealing incision.

20. The method of claim 18 wherein step (c) involves attaching a suture between said drainage shunt and said cornea portion.
